(19) United States
(12) Patent Application Publication
(10) Pub. No.: US 2003/0190657 A1
(43) Pub. Date: Oct. 9, 2003

(54) Rafiki model and map to the genetic code

(76) Inventor: Mark P. White, Bloomington, IN (US)

Correspondence Address: Michael B. McNeil, Liell & McNeil Attorneys PC, P.O. Box 2417, Bloomington, IN, 47402, US

(21) Appl. No.: 10/396,670

(22) Filed: Mar. 25, 2003

Related U.S. Application Data
(60) Provisional application No. 60/367,653, filed on Mar. 26, 2002.
(60) Provisional application No. 60/415,623, filed on Oct. 2, 2002.
(60) Provisional application No. 60/419,919, filed on Oct. 21, 2002.
(60) Provisional application No. 60/426,295, filed on Nov. 14, 2002.
(60) Provisional application No. 60/439,344, filed on Jan. 10, 2003.

(51) Int. Cl. C12Q 1/68; G06F 19/00; G01N 33/48; G01N 33/50

(52) U.S. Cl. 435/6; 702/20

(57) ABSTRACT

A map represents a network of relationships among a first set of symbols and a second set of symbols. The first set of symbols can be genetic base codes, which are each one of four types. The second set of symbols can represent the twenty standard amino acids and stops that occur in almost all life on the planet earth. The map can be embedded in computer code, reflected by an electronic database or visually presented on a substrate, which can include color and a dodecahedral logic structure projected onto a globe. The globe can be a sphere, a dodecahedron, an icosahedron, a soccer ball (Archimedian solid), or an equivalent. The network of relationships reflected by the map can be used to decode a sequence of genetic base codes into a sequence of amino acids in a protein.

### RELATION TO OTHER PATENT APPLICATIONS

This application claims the benefit of provisional application Nos. 60/367,653; 60/415,623; 60/419,919; 60/426,295; and 60/439,344 filed on Mar. 26, 2002, Oct. 2, 2002, Oct. 21, 2002, Nov. 14, 2002 and Jan. 10, 2003, respectively.

### TECHNICAL FIELD

The present invention relates generally to maps for representing relationships among sets of symbols, and more particularly to a map representing relationships among genetic base codes and amino acids that occur in nature.

### BACKGROUND

DNA includes sequences of the nucleic acids adenine (A), guanine (G), cytidine (C), and thymidine (T). MRNA uses the same four block system with the exception that thymidine (T) is replaced with uracil (U). These blocks are often referred to as genetic base codes and they represent the letters of the genetic alphabet. When a sequence of genetic base codes are processed, meaning is passed to TRNA and then to amino acids by grouping three base codes together to form a codon. Since there are sixty-four ways to order a subset of three out of an available four, the genetic language can be thought of as having sixty-four words or codons.

Over time, scientists have come to recognize that almost all life on this planet is based on twenty standard amino acids. When genetic base codes are processed, each codon is identified with one of the standard twenty amino acids. Thus, when the genetic base codes are processed, each codon is processed sequentially to assign one of the twenty amino acids as the next building block in the construction of a protein. If one is given a sequence of codons, one can predict precisely the sequence of amino acids that will appear in the resulting protein. The twenty standard amino acids include isoleucine, phenylalanine, valine, leucine, methionine, tryptophan, alanine, glycine, cysteine, tyrosine, proline, threonine, serine, histidine, glutamate, asparagine, glutamine, aspartate, lysine, and arginine. Although there are many more amino acids that stabily exist in the universe, almost all life on earth utilizes that same twenty amino acids. In addition, the standard twenty amino acids also naturally occur in two different mirror image forms, often called L-type and D-type. It is important to note that all of the standard twenty amino acids are of the L-type, despite the fact that the D-type also naturally occur.

Most good biochemistry textbooks include a table or grid that allows one to identify a codon and the individual amino acid assigned to that codon. These assignment tables can come in a variety of forms, but they all suffer from an inability to accurately represent the network of relationships that exist in nature among genetic base codes and the standard set of twenty amino acids. In fact, because conventional wisdom avoids the question, there is little agreement as to whether a network of relationships actually even exists.

The present invention is directed to an improved presentation of the relationships among genetic base codes and amino acids, as well as elucidating a network of relationships among these genetic building blocks.

### SUMMARY OF THE INVENTION

In one aspect, the invention includes a method of decoding a code having a sequence of symbols, with each symbol being from a first set of from 12-23 symbols. The first set of symbols are linked in a network to a second set of at least 20 translated symbols. Each member of the first set of symbols is linked to several members of the second set of translated symbols. The sequence of symbols is translated into a sequence of translated symbols using the network.

In another aspect, a map includes a first set of symbols and a second set of symbols. A set of less than seven members of the second set of symbols are mapped to a set of three members of the first set of symbols. Relationships between the first and second sets of symbols represent relationships between genetic base codes and amino acids that occur in nature.

In another aspect, a map includes twenty assignments mapped to subsets of twenty amino acids and stops. Four of the subsets have a primary pattern, twelve of the subsets have a secondary pattern, and four of the subsets have a tertiary pattern.

In still another aspect, a map includes at least twenty subsets mapped to each other in a network of relationships. Each of the subsets being representative of one of twenty amino acids and stops. At least one of the subsets represent an amino acids corresponding to a plurality of base code codons.

In another aspect, a map includes a first set of symbols mapped to a second set of symbols. The first set of symbols include less than twenty-four members, which are each one of four different types. The second set of symbols includes at least twenty different members. Each combination of three members of the first set of symbols are mapped to at least one member of the second set of symbols. At least one of the combinations is mapped to a plurality of different members of the second set of symbols.

In still another aspect, a method of determining an assignment relationship between genetic base codes and amino acids includes a step of mapping a network of relationships among genetic base codes and amino acids. One of an amino acid and a group of three adjacent base codes are identified in the network. A group of three adjacent base codes or an amino acid, respectively, are then read from the network.

In still another aspect, a map includes genetic base codes arranged in a pattern corresponding to at least a portion of a regular solid. Amino acids are mapped in a predetermined relationship with respect to the genetic base codes such that each ordered combination of three genetic base codes are mapped to one of the amino acids. The predetermined relationship reflects a genetic base code-amino acid assignment relationship that occurs in nature.

### BRIEF DESCRIPTION OF THE DRAWINGS

This patent or application file contains at least one drawing executed in color. Copies of this patent or patent application publication with color drawings will be provided by the office upon request and payment of the necessary fee.

A variety of products, including versions of the maps presented in the Figures described below should be available from the patent owner when this document is published by contacting Rafiki, Inc. at 3309 Mulberry Court, Bloomington, Ind. 47401, and at the internet website codefun.com.

FIG. 1 is a genetic codon assignment table in which the amino acids are stratified by water affinity.

FIG. 2 is a perspective view of a generalized map of the Rafiki model illustrated in the context of an unfolded dodecahedron.

FIG. 3 is a perspective view of a genetic code map according to the present invention.

FIG. 4 is a perspective view of the map of FIG. 3 with the addition of the color coded water affinity symbols from the table of FIG. 1.

FIG. 5 is a set of related maps according to another aspect of the present invention.

FIG. 6 is a perspective view of a map according to still another aspect of the present invention in which the triangles of FIG. 5 are joined together into an unfolded icosahedron.

FIG. 7 includes twenty different views of a spherical map according to still another aspect of the present invention.

FIG. 8 is an illustration showing the patterns produced by the top row of views from FIG. 7.

FIG. 9 is a perspective view of a map according to still another aspect of the present invention in the form of an unfolded soccer ball pattern.

### DETAILED DESCRIPTION

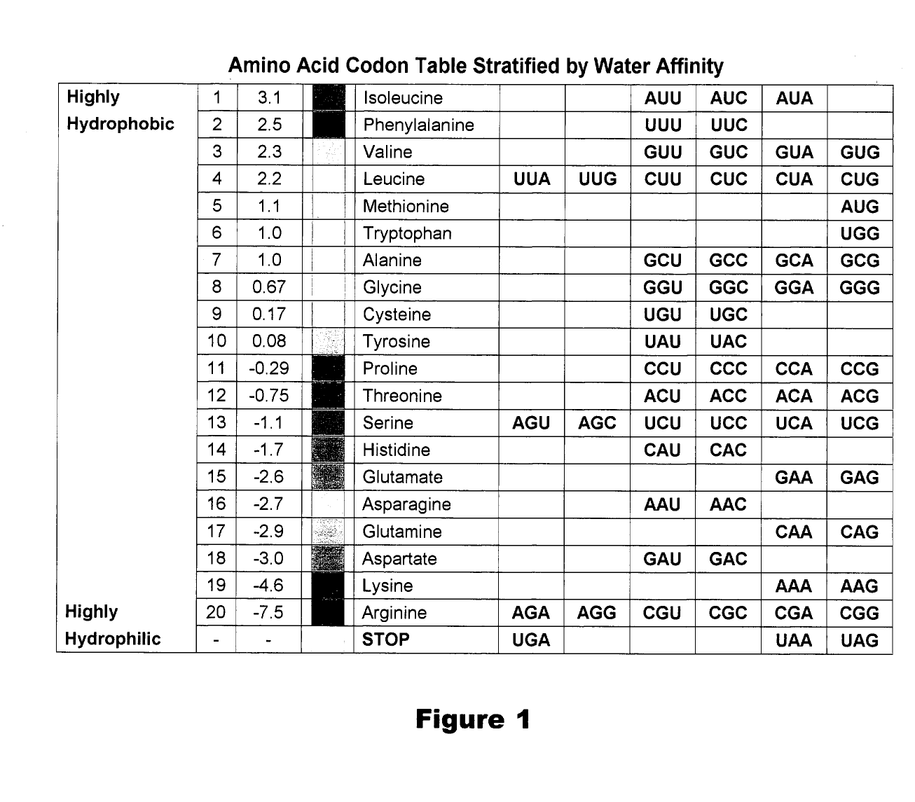

Referring to FIG. 1, a table lists all twenty standard amino acids and their codon assignments. The amino acids are listed from top to bottom according to water affinity, which is qualified in the first column and quantified in the second column. The third column lists the numerals one through twenty, and the fourth column provides a spectrum of color symbols to represent water affinity. In this color scheme, the highly hydrophobic amino acids, isoleucine and phenylalanine are generally reddish in color, whereas the highly hydrophilic amino acids leucine and arginine are more bluish in color. Red is generally water hating, while blue is generally water loving. The fifth column lists the twenty amino acids by name. The sixth and seventh columns are devoted to those three amino acids, leucine, serine and arginine, that each have six different recognized codon assignments. The eighth, ninth, tenth and eleventh columns include all other codons that end in U, C, A or G, respectively. The stop codons UGA, UAG and UAA are arranged on the bottom row of the table according to these conventions.

Of note is the fact that the table of FIG. 1 needs as many as 192 nucleic acids to express 64 different codons. Some known prior art nucleic acid/amino acid assignment tables can include specific grids with as few as twenty-four nucleic acids arranged in a pattern designed to identify all sixty-four commonly recognized codons. However, all of these prior art tables and maps suffer from a subtle but important disadvantage in their ability to present the relationships among nucleic acids and amino acids in a simplified, and maybe more importantly, an unbiased manner. In an effort to reach this goal, a map(s) of the present invention will be structured in a way in which all nucleic acids are treated equally and efficiently. The term “map”, as used in this patent document, means more than a visible image on a physical substrate. The term “map” can include a projection on computer monitor, an electronic database that is neither visible nor tangible but does have a predetermined network of relationships among the members of the database, or possibly even computer code with the invention's network of relationships incorporated therein.

The invention can function with as few as twelve nucleic acids, which can be three each of the four types for most life forms. Yet with this minimum of only twelve nucleic acids to spend, but we must achieve at least twenty assignments, which are correlated to the twenty amino acids. This can be accomplished by spending each nucleic acid five times in forming the identical triplet nucleic acid codons, which each correlate to a single amino acid. The following table illustrates this concept. However, we shall use generalized symbols at this time before inserting simple symbols more commonly representative of nucleic acids. Let A1-A12 represent a set of symbols that could represent the twelve nucleic acids for our new map. Let B1-B20 represent 20 different assignments. Symbols separated by commas are unordered; symbols separated dashes are ordered.

A1=(B1, B2, B3, B4, B5)

A2=(B1, B2, B6, B7, B8)

A3=(B2, B3, B8, B9, B10)

A4=(B3, B4, B10, B11, B12)

A5=(B4, B5, B12, B13, B14)

A6=(B1, B5, B6, B14, B15)

A7=(B9, B10, B11, B16, B17)

A8=(B7, B8, B9, B17, B18)

A9=(B6, B7, B15, B18, B19)

A10=(B13, B14, B15, B19, B20)

A11=(B11, B12, B13, B16, B20)

A12=(B16, B17, B18, B19, B20)

It should be noted that the inversion of this thinking is that each nucleic acid participates in multiple assignments. This theory suggests that the assignment process may have involved nucleic acids and amino acids simultaneously converging on codons. In other words, the codons could not have existed in any meaningful way before this “mystical” assignment lottery. Since we now appear to require an inter-related network of nucleic acids, we are driven to assume substrate neutrality. This means that if a nucleic acid, alanine for instance, can be plugged into A1, it can be plugged into any or all of the other eleven symbols as well. Substrate neutral systems of triplets have six permutations as follows:

Permutation#1, P1 =1, 2, 3

Permutation#2, P2 =2, 3, 1

Permutation#3, P3 =3, 1, 2

Permutation#4, P4 =1, 3, 2

Permutation#5, P5 =3, 2, 1

Permutation#6, P6 =2, 1, 3

This implies that in our new model we must accept that their are six distinguishable permutations of all possible nucleic acids, including seemingly trivial cases such as (adanine, adanine, adanine). Each amino acid assignment represents a collection of all permutations of the three nucleic acids that are related to it.

B1=ΣP(A1, A6, A2)

B2=ΣP(A1, A2, A3)

B3=ΣP(A1, A3, A4)

B4=ΣP(A1, A4, A5)

B5=ΣP(A1, A5, A6)

B6=ΣP(A2, A6, A9)

B7=ΣP(A2, A9, A8)

B8=ΣP(A2, A8, A3)

B9=ΣP(A3, A8, A7)

B10=ΣP(A3, A7, A4)

B11=ΣP(A4, A7, A11)

B12=ΣP(A4, A11, A5)

B13=ΣP(A5, A11, A10)

B14=ΣP(A5, A10, A6)

B15=ΣP(A6, A10, A9)

B16=ΣP(A7, A12, A11)

B17=ΣP(A7, A8, A12)

B18=ΣP(A8, A9, A12)

B19=ΣP(A9, A10, A12)

B20=ΣP(A10, A11, A12)

There is a potential danger here in failing to recognize the meaning of any assignment within this system. We started with only twenty required assignments, because that is what the empirical evidence suggested that we do. But our assignment process immediately yielded multiple potential meanings as to each assignment triplet depending on its context within the model. For instance, notice that the nucleic acid represented by A1 is related to five of the assignments, and for each of these, A1 is the initial base in the assignment permutation exactly twice.

A1=(B1, B2, B3, B4, B5)

B1=(A1-A6-A2), (A6-A2-A1), (A2-A1-A6), (A1-A2-A6), (A2-A6-A1), (A6-A1-A2)

This holds true for all of the twelve nucleic acids and their related assignments, so each base code is a primary initiator of five codons and a secondary initiator of five codons. Therefore, there are sixty primary initiators and sixty secondary initiators. We will assign each permutation a label so that we can demonstrate each symbol's role as initiator, such as C1=(A1-A6-A2) and C61=(A1-A2-A6).

| Primary Initiators | Secondary Initiators |
|---|---|
| A1 = (C1, C2, C3, C4, C5) | A1 = (C61, C62, C63, C64, C65) |
| A2 = (C6, C7, C8, C9, C10) | A2 = (C66, C67, C68, C69, C70) |
| A3 = (C11, C12, C13, C14, C15) | A3 = (C71, C72, C73, C74, C75) |
| A4 = (C16, C17, C18, C19, C20) | A4 = (C76, C77, C78, C79, C80) |
| A5 = (C21, C22, C23, C24, C25) | A5 = (C81, C82, C83, C84, C85) |
| A6 = (C26, C27, C28, C29, C30) | A6 = (C86, C87, C88, C89, C90) |
| A7 = (C31, C32, C33, C34, C35) | A7 = (C91, C92, C93, C94, C95) |
| A8 = (C36, C37, C38, C39, C40) | A8 = (C96, C97, C98, C99, C100) |
| A9 = (C41, C42, C43, C44, C45) | A9 = (C101, C102, C103, C104, C105) |
| A10 = (C46, C47, C48, C49, C50) | A10 = (C106, C107, C108, C109, C110) |
| A11 = (C51, C52, C53, C54, C55) | A11 = (C111, C112, C113, C114, C115) |
| A12 = (C56, C57, C58, C59, C60) | A12 = (C116, C117, C118, C119, C120) |

These permutations (C1-C120) represent codons, so that we can substitute them into the relationship between assignments and nucleic acid permutation sets, rounding out our comprehensive set of interrelated assignments.

B1=(C1, C28, C9, C61, C88, C69)

B2=(C2, C8, C14, C62, C68, C74)

B3=(C3, C13, C19, C63, C73, C79)

B4=(C4, C18, C24, C64, C78, C84)

B5=(C5, C23, C29, C65, C83, C89)

B6=(C10, C27, C41, C70, C87, C10)

B7=(C6, C45, C37, C66, C105, C97)

B8=(C7, C36, C15, C67, C96, C75)

B9=(C11, C40, C32, C71, C100, C92)

B10=(C12, C31, C20, C72, C91, C80)

B11=(C16, C35, C52, C76, C95, C112)

B12=(C17, C51, C25, C77, C121, C95)

B13=(C21, C55, C47, C81, C115, C107)

B14=(C22, C46, C30, C82, C106, C90)

B15=(C26, C50, C42, C86, C110, C102)

B16=(C34, C56, C53, C94, C116, C113)

B17=(C33, C39, C57, C93, C99, C127)

B18=(C38, C44, C58, C98, C104, C118)

B19=(C43, C49, C59, C103, C109, C119)

B20=(C48, C54, C60, C108, C114, C120)

C1 = A1 − A6 − A2
C2 = A1 − A2 − A3
C3 = A1 − A3 − A4
C4 = A1 − A4 − A5
C5 = A1 − A5 − A6
C6 = A2 − A9 − A8
C7 = A2 − A8 − A3
C8 = A2 − A3 − A1
C9 = A2 − A1 − A6
C10 = A2 − A6 − A9
C11 = A3 − A8 − A7
C12 = A3 − A7 − A4
C13 = A3 − A4 − A1
C14 = A3 − A1 − A2
C15 = A3 − A2 − A8
C16 = A4 − A7 − A1
C17 = A4 − A11 − A5
C18 = A4 − A5 − A1
C19 = A4 − A1 − A3
C20 = A4 − A3 − A7
C21 = A5 − A11 − A11
C22 = A5 − A10 − A6
C23 = A5 − A6 − A1
C24 = A5 − A1 − A4
C25 = A5 − A4 − A11
C26 = A6 − A10 − A9
C27 = A6 − A9 − A2
C28 = A6 − A2 − A1
C29 = A6 − A1 − A5
C30 = A6 − A5 − A10
C31 = A7 − A4 − A3
C32 = A7 − A3 − A8
C33 = A7 − A8 − A12
C34 = A7 − A12 − A11
C35 = A7 − A11 − A4
C36 = A8 − A3 − A2
C37 = A8 − A2 − A9
C38 = A8 − A9 − A12
C39 = A8 − A12 − A7
C40 = A8 − A7 − A3
C41 = A9 − A2 − A6
C42 = A9 − A6 − A10
C43 = A9 − A10 − A12
C44 = A9 − A12 − A8
C45 = A9 − A8 − A2
C46 = A10 − A6 − A5
C47 = A10 − A5 − A11
C48 = A10 − A11 − A12
C49 = A10 − A12 − A9
C50 = A10 − A9 − A6
C51 = A11 − A5 − A4
C52 = A11 − A4 − A7
C53 = A11 − A7 − A12
C54 = A11 − A12 − A10
C55 = A11 − A10 − A5
C56 = A12 − A11 − A7
C57 = A12 − A7 − A8
C58 = A12 − A8 − A9
C59 = A12 − A9 − A10
C60 = A12 − A10 − A11
C61 = A1 − A2 − A6
C62 = A1 − A3 − A2
C63 = A1 − A4 − A3
C64 = A1 − A5 − A4
C65 = A1 − A6 − A5
C66 = A2 − A8 − A9
C67 = A2 − A3 − A8
C68 = A2 − A1 − A3
C69 = A2 − A6 − A1
C70 = A2 − A9 − A6
C71 = A3 − A7 − A8
C72 = A3 − A4 − A7
C73 = A3 − A1 − A4
C74 = A3 − A2 − A1
C75 = A3 − A8 − A2
C76 = A4 − A11 − A7
C77 = A4 − A5 − A11
C78 = A4 − A1 − A5
C79 = A4 − A3 − A1
C80 = A4 − A7 − A3
C81 = A5 − A10 − A11
C82 = A5 − A6 − A10
C83 = A5 − A1 − A6
C84 = A5 − A4 − A1
C85 = A5 − A11 − A4
C86 = A6 − A9 − A10
C87 = A6 − A2 − A9
C88 = A6 − A1 − A2
C89 = A6 − A5 − A1
C90 = A6 − A10 − A5
C91 = A7 − A3 − A4
C92 = A7 − A8 − A3
C93 = A7 − A12 − A8
C94 = A7 − A11 − A12
C95 = A7 − A4 − A11
C96 = A8 − A2 − A3
C97 = A8 − A9 − A2
C98 = A8 − A12 − A9
C99 = A8 − A7 − A12
C100 = A8 − A3 − A7
C101 = A9 − A6 − A2
C102 = A9 − A10 − A6
C103 = A9 − A12 − A10
C104 = A9 − A8 − A12
C105 = A9 − A2 − A8
C106 = A10 − A5 − A6
C107 = A10 − A11 − A5
C108 = A10 − A12 − A11
C109 = A10 − A9 − A12
C110 = A10 − A6 − A9
C111 = A11 − A4 − A5
C112 = A11 − A7 − A4
C113 = A11 − A12 − A7
C114 = A11 − A10 − A12
C115 = A11 − A5 − A10
C116 = A12 − A7 − A11
C117 = A12 − A8 − A7
C118 = A12 − A9 − A8
C119 = A12 − A10 − A9
C120 = A12 − A11 − A10

Although we achieved a potential 192 to 12 reduction in nucleic acids, we also note a peculiar increase in the number of required permutations from 64 to 120. This is due to the model's inability to distinguish between seemingly trivial permutations at the triplet level; however, this new model is not a two dimensional, one to one, sequestering grid; it is a multi-dimensional interrelation network, which we can call an identity network.

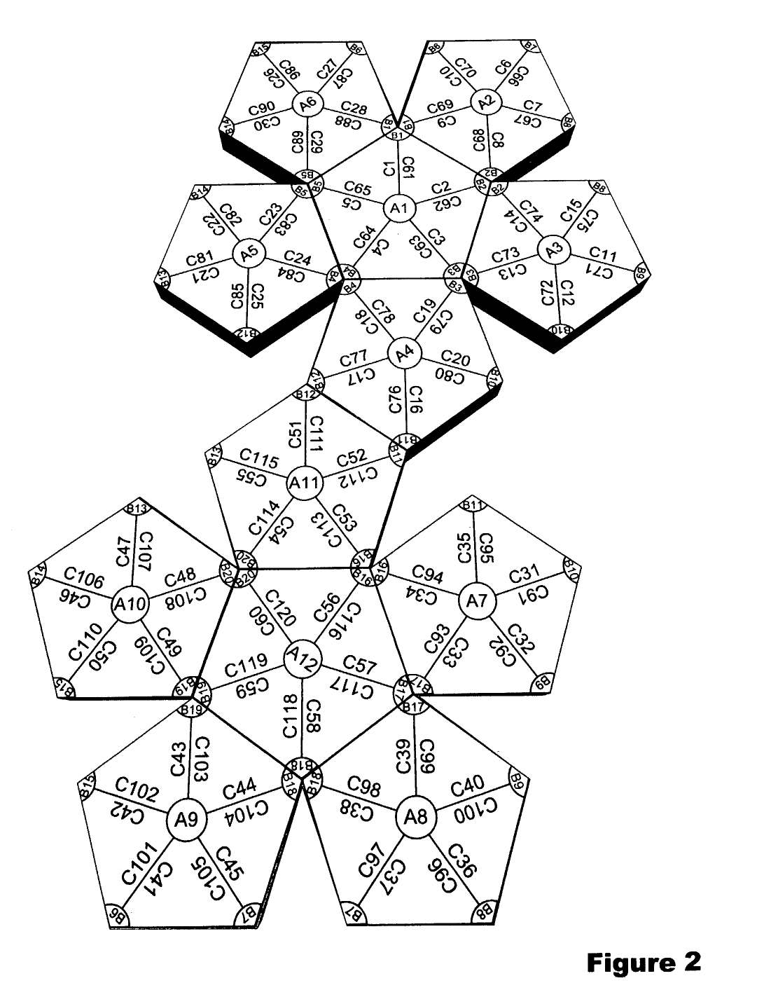

One seemingly glaring drawback to this model is that, unlike the grids normally used to demonstrate the conventional model of the genetic code, the identity network does not lend itself easily to a two dimensional schematic representation. However, what is lacks in two dimensions, it more than makes up for in three dimensions. We could view the network primarily from the perspective of amino acids, or primarily from the perspective of nucleic acids. The former requires twenty sub-units and the later only twelve. Therefore, choosing the most efficient we first generate a dodecahedron rather than an icosahedron, but they are dual to each other. In fact, the concept can be interpreted as a sphere, but polyhedrons are often more effective, given a flat starting substrate, such as paper. When all of these relationships are combined, we arrive at the generalized map shown in FIG. 2. Those skilled in the art will recognize that a computer could be programmed to represent the relationships reflected by the map of FIG. 2, and that programming code or an electronic database would be a “map” according to the present invention.

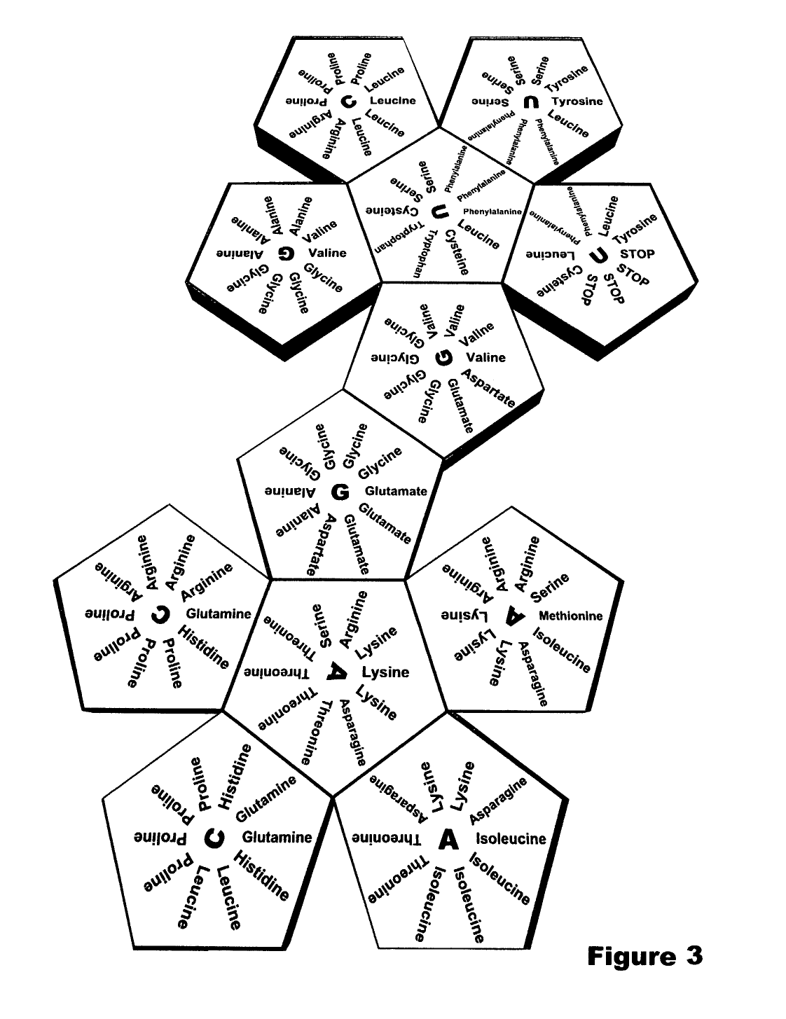

A full appreciation of the relationships in this identity network require that the diagram be cut and folded into a dodecahedron. When we substitute a different set of symbols representing nucleic acids in for A1-A12, and a second set of symbols representing the twenty standard amino acids and stops for the C1-C120 symbols we arrive at the map shown in FIG. 3. Again, the map of FIG. 3 best reflects all of the interrelationships when folded into a globe, such as a dodecahedron. Nevertheless, those skilled in the art will realize that the present invention can be presented in many visually different ways (two dimensions, three dimensions, projections, etc.) so long as the network of relationships is maintained. The map is read in the case of identifying an amino acid associated with the codon by identifying an ordered set of three nucleic acids that make up any particular codon. One can quickly see that these three nucleic acids can be thought of as forming a triangle on the map of FIG. 3. The particular amino acid is identified by identifying the first nucleic acid in the specific codon and then moving along the leg of the triangle toward the second nucleic acid in that codon. The first amino acid within the triangle that is encountered in this process represents the assignment of that particular codon. For instance, the codon GAG corresponds to glutamate, but the codon GGA corresponds to glycine. In the map of FIG. 3, the MRNA base code U is used; those skilled in the art will appreciate that the DNA base code T could be substituted in the place of U without otherwise altering the map of the present invention.

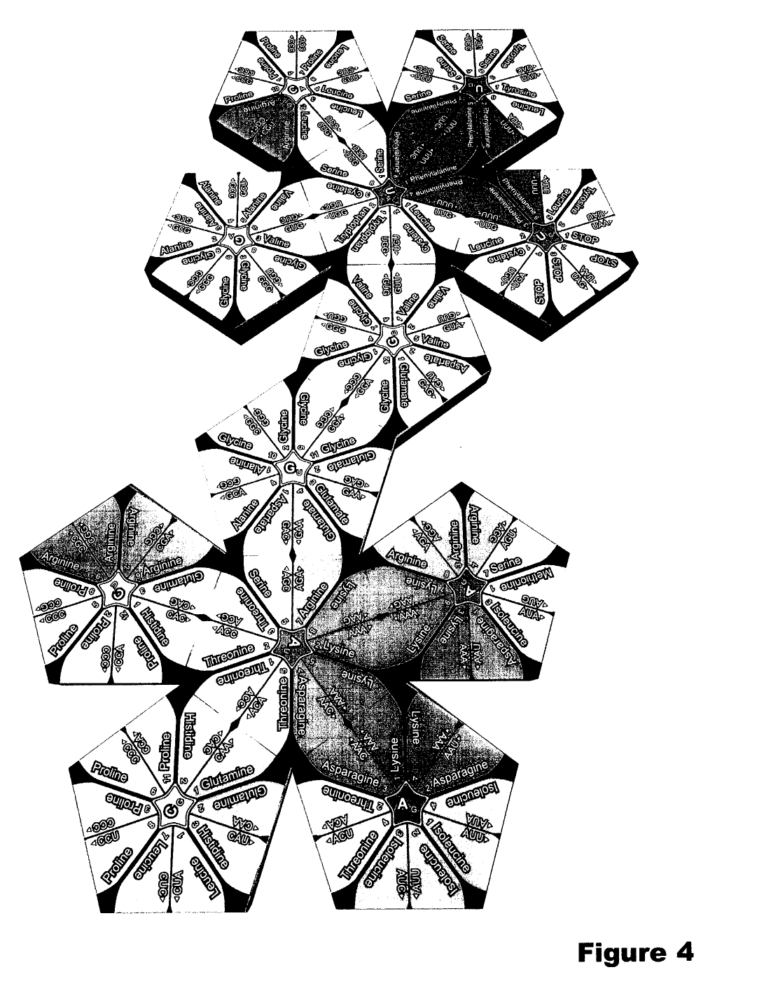

Referring now to FIG. 4, the map of FIG. 3 has been rearranged and the color symbols from the table of FIG. 1 have been added. This map is read in a similar manner. For instance, if one is to determine a codon for a particular amino acid, an amino acid is identified on the map. Next, one identifies the three nucleic acids associated with that amino acid. For instance, one can determine that the codon for methionine is AUG. In another example, one of the versions of serine corresponds to the codon AGC. The map of FIG. 4 also brings forward other aspects of the generalized map of FIG. 2. In particular, each appearance of each amino acid is identifiably different from the other appearances of that same amino acid. For instance, the map identifies Lysine 1-8. In addition, each of the twelve nucleic acid base codes are identifiably different from one another. In this map, this is accomplished by giving each nucleic acid a subscript representing the nucleic acid on an opposite face of the dodecahedron. For instance G_{U} is opposite from U_{G}. Each base code is positioned in a star of one of the four colors, which are blue (A), green (C), yellow (G) and red (U). This color convention for the nucleic acids is carried through on the other maps, and can be thought of representing water affinity with respect to the amino acids lysine, proline, glycine and phenanalinine, respectively, with which they are most closely associated. Finally, each codon is also shown with an arrow to assist in reading assignment information from the map.

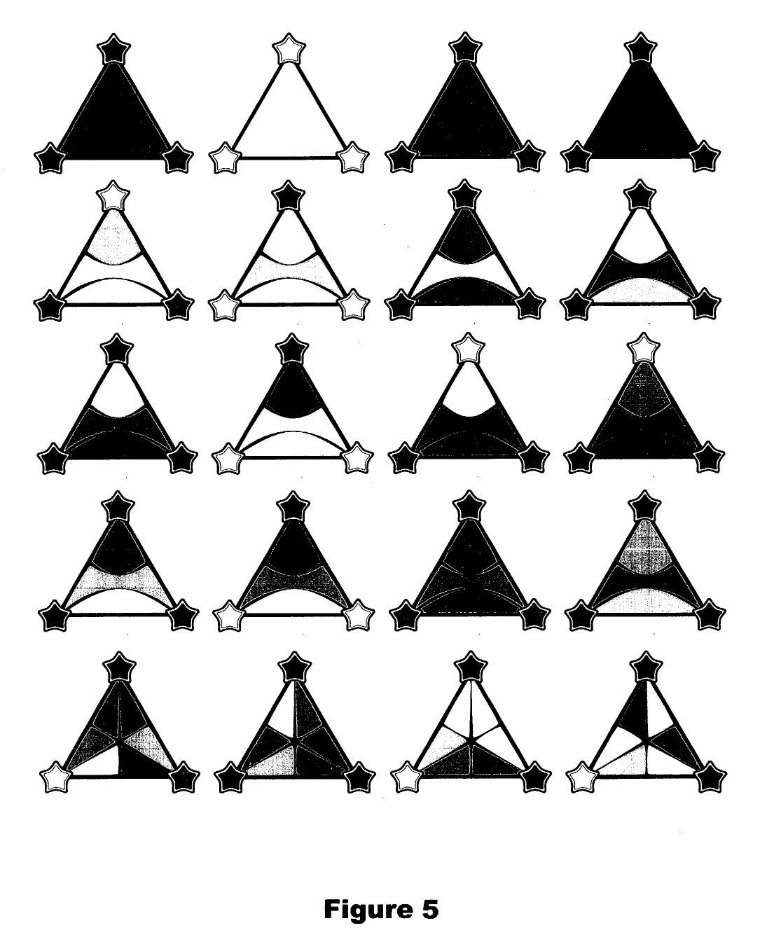

Patterns emerge from the maps, and these patterns represent relationships within the genetic code. Although nucleic acids have become equal, triplets have become decidedly unequal. There are now three cases of triplets: primary, secondary and tertiary. When each of the triangular assignments or subsets demonstrated by the map of FIG. 4 is separated from the others and laid out in a grid, we arrive at the set of maps of FIG. 5. Each of the colored stars represents a genetic base code or nucleic acid. In particular, red stars correspond to U, yellow to G, green to C and blue stars to A. The top row of triangles in FIG. 5 can be considered a primary pattern, the next three rows can be considered the secondary pattern, and the last row representing the tertiary pattern. Recalling, each of the colors within the triangles represent one of the twenty different shades of color presented in the table of FIG. 1, with the exception that the stops are now colored white or a light lavender color. In the case of each primary pattern, there is one amino acid associated with each triplet. These include phenylalanine (red), glycine (yellow), proline (green) and lysine (blue). Each of the secondary patterns correspond to three amino acids, with the exception that the (A, A, U) assignment includes two amino acids and a stop codon. Each of the tertiary patterns represent six amino acids, with the exception that the (G, U, A) assignment includes four amino acids and two stop codons.

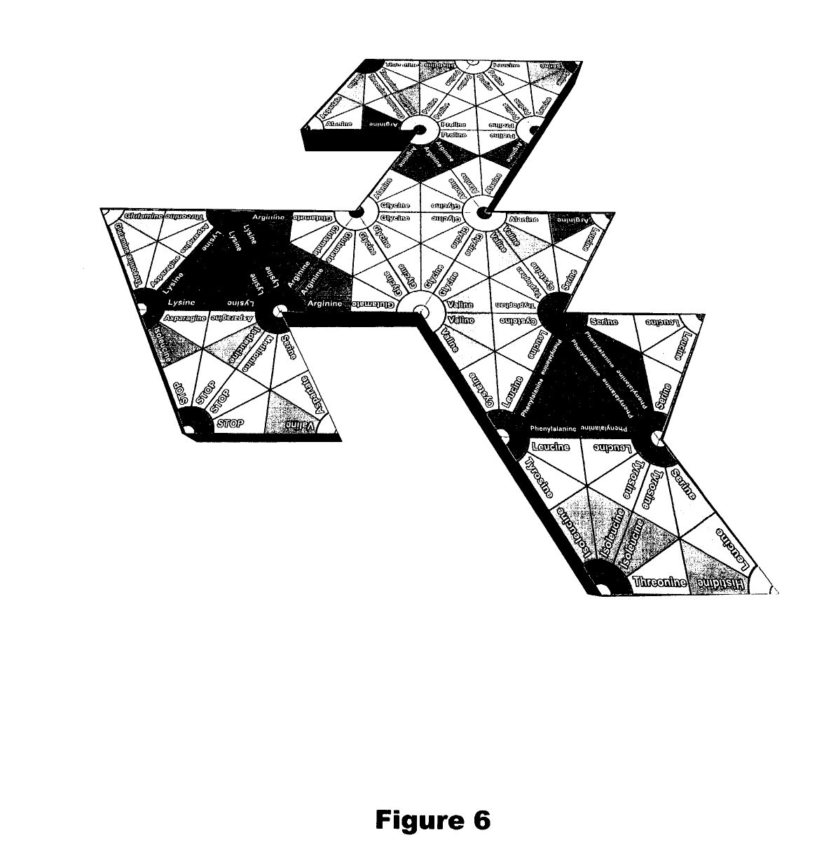

When the triangular assignment subsets of FIG. 5 are joined to one another, we arrive at the unfolded icosahedron map similar to that of FIG. 6. FIG. 6 also has a feature that was not a part of FIG. 5, but instead is a feature carried forward from the generalized map of FIG. 2. In particular, each of the base code nucleic acids is individually identifiable with regard to the eleven other nucleic acids. In the general case of FIG. 2, this was accomplished merely by numbering each of the dodecahedron faces with A1-A12. In the case of FIG. 6, each nucleic acid is uniquely identified by the nucleic acid that is opposite to it when the map of FIG. 6 is folded into an icosahedron. For instance, the A_{U} is directly opposite from the U_{A} nucleic acid. Using colors, this enables each of the twelve base code nucleic acids to be readily and uniquely identified. When folded into an icosahedron, the primary pattern faces are distributed in a tetrahedral pattern. In addition, the tertiary faces are also distributed in a tetrahedral pattern, which is the dual to the tetrahedron of the primary patterns. Each edge of each of the primary and tertiary patterns is contiguous with a different subset of three secondary pattern assignments.

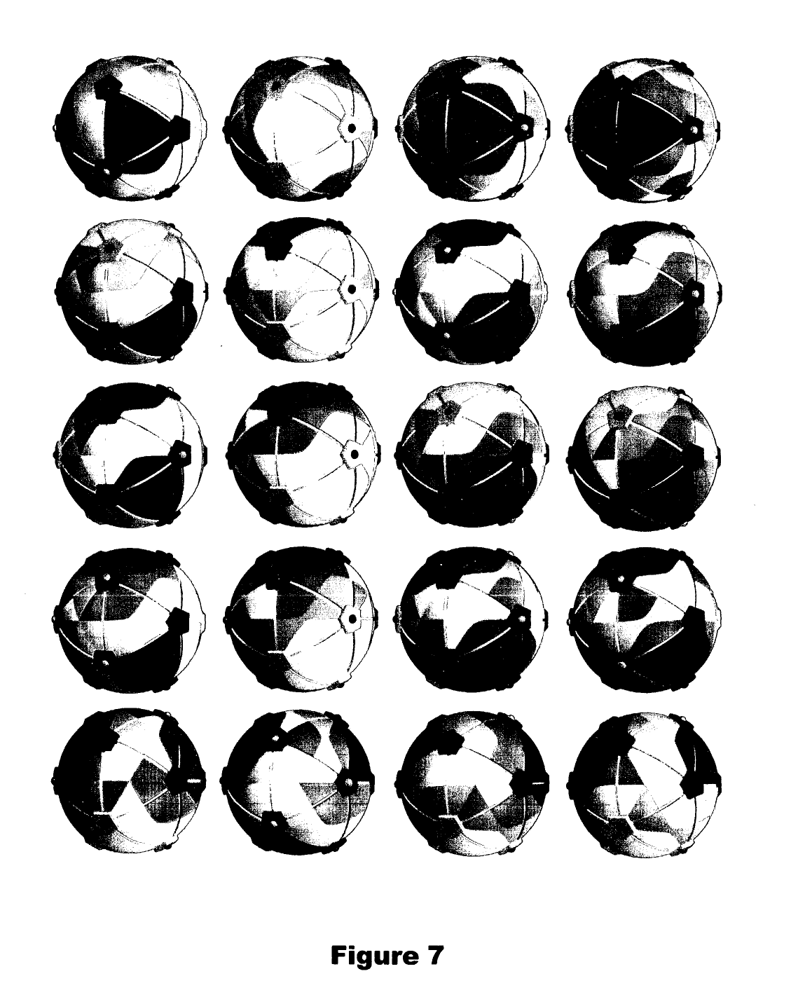

When the map of FIG. 6 is folded and projected onto the surface of the sphere, we arrive at a map similar to FIG. 7. In FIG. 7, a sphere is broken up to include a substantial variety of different shaped contiguous regions that are each colored to correspond to one of the amino acids according to the color coded symbols first presented in the table of FIG. 1. There are a total of 64 regions on the globe of FIG. 7. Each region represents one amino acid or stop; however, some of the regions are larger than others. This reflects that some amino acids, argine for instance, span an area that stretches across several codons. Some amino acids, such as serine, have several regions that are isolated from one another. These regions can be thought of as subsets of the twenty amino acids and stops. Each triangle is defined by three pentagons that are color coded as per the base code color symbols presented earlier. In addition, each of the base code nucleic acids is uniquely identifiable due to the color dot at its center which corresponds to the nucleic acid on the opposite side of the sphere or globe from any given nucleic acid. Those skilled in the art will quickly recognize that the twenty faces of the sphere of FIG. 7 correspond to the arrangement of the twenty triangles in the set of maps of FIG. 5.

We started with a rearrangement of the known prior art codon-amino acid assignment table that some consider to be a linear phenomenon that is the result of an arbitrary and meaningless accident frozen in time. The present invention rearranges the data into three dimensions to reveal previously unseen patterns. Preferably, color is used to provide a more ideal perception of the patterns. These patterns, like all patterns, can be assigned meaning. This has opened a whole new arena for an investigation of patterns, which we call the network space. In a network space, several curious things happened. Nucleic acids equalized, triplets became combinatorial, and codons became differentiated based on their generative triplet and location within that triplet. The reason that networking the assignment table generates patterns that correlate across identifiable parameters, such as codon differentiation, is because the assignment logic is not linear, and is not arbitrary, as the dogma of the prior art has suggested. The assignment logic is only a part of a larger system that is in fact a network that was not previously recognized.

The Rafiki model treats the genetic code as a network of inter-related components. Nucleic acids are inter-related with other nucleic acids, other triplets, codons, TRNA and amino acids. Amino acids seem to cooperate with each other by distributing themselves uniformly across the network of nucleic acids. The functional groups seem to play a role with respect to water affinity in the overall distribution of codon assignments. If this is true, there must be some additional information hidden in the genetic code. From the new corrective view permitted by the Rafiki model, I have found that overlooked information in the genetic code appears related to stereochemistry. In other words, the peptide bond between adjacent amino acids is a quantified entity that is completely described in an overlapping portion of the code. Thus, amino acid assignment is only a portion of the code, namely the context for the peptide bond. The definition of these peptide bonds describes the primary structure of a protein. Therefore, it is primary structure, not merely primary sequence as previously believed, that dictate secondary structure. It is believed that the peptide bond can be quantized according to the participants and possibly into as many as six categories, which include cis and trans configurations. Each of cis and trans can have three configurations of its own, namely, Ramachandran one, two and three. The stereochemistry is suggested in the least by the same amino acid appearing at different locations on the map. For instance, serine-1 would be attached to a previous amino acid in one orientation, while serine-5 reflects serine attached in a different orientation in the polypeptide chain.

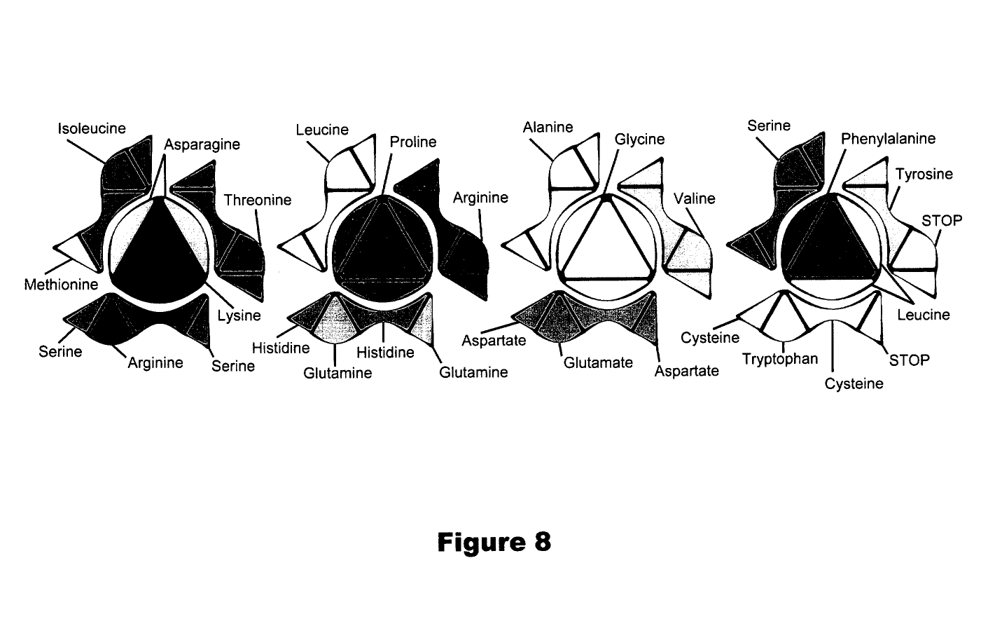

Those studying the maps of the present invention will recognize that each amino acid occupies one or more different regions on the globe. In some cases, such as arginine, threonine, leucine, alanine, valine and serine, these regions span across four contiguous triangular faces. These different regions are extracted from the globe of FIG. 7 and illustrated around the primary pattern faces to reveal four flower like patterns as shown in FIG. 8. While most of the amino acids occupy only a single region, several occupy two separate regions, such as cysteine, aspartate, glutamine and histidine. Others occupy as many as three separate regions. These include leucine and serine. When one amino acid spans across several contiguous triangular faces, this reveals that the codons are related to define these regions and that contiguous amino acids are likely related to one another. In prior art versions of the genetic code assignment table, these relationships among nucleic acids, codons and amino acids were not evident.

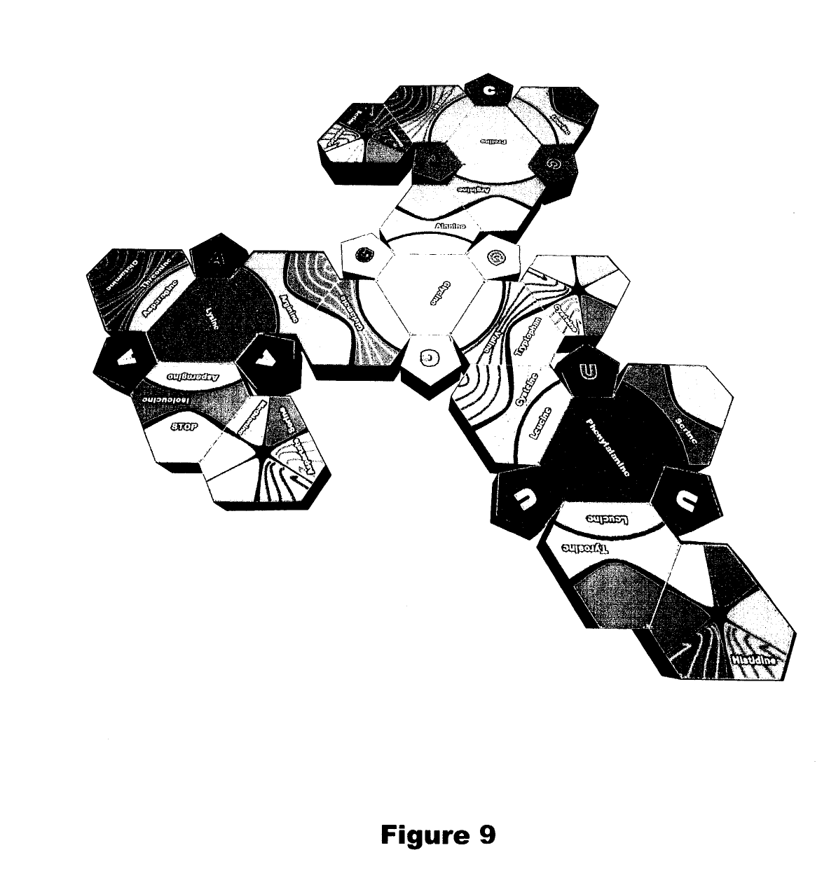

When the pattern of FIG. 7 is again adjusted, we can arrive at the “soccer ball” pattern shown in FIG. 9. In this map of the present invention, several of the amino acid regions are colored with a primary color and stripes to reduce the number of required colors down from twenty. In addition, this strategy allows the colors that are naturally close in shade to one another to be more easily differentiated based upon color alone. For instance, valine is orange with red stripes. Glutamine is dark blue with light blue stripes. Histidine is light blue with green stripes. Threonine is dark green with light green stripes. Cysteine is yellow with green stripes, and methionine is yellow with orange stripes. Glutamate is green with light blue stripes. Those skilled in the art will appreciate that the pattern of FIG. 9 can be constructed into a ball using conventional soccer ball manufacturing techniques. In the map of FIG. 9, each of the nucleic acid base codes are differentiated from one another based upon the color used to identify the nucleic acid. For instance, G is generally yellow, but each of the three letter G's on the map is identified with a blue G, a green G and a red G. As discussed earlier, the red G is directly opposite on the globe from the yellow U. All of the other nucleic acids share a similar relationship to an opposite nucleic acid on the opposite side of the globe. When folded into a globe, each colored region preferably includes a word identifying the particular amino acid for that color. This better enables map reading without need to reference the table of FIG. 1.

Information theory is about accounting for possibilities. We try to identify all possible conditions, those that activate and those that repress. Assignment of individual amino acids to combinations of nucleic acids must also operate on two levels of constraint. The first is the set of all possible combinations of nucleic acids that can be present, and the second is the set of all possible combinations of nucleic acids that can be absent. The first set has sixty-four members and the second set has twenty members. If the assignment process is to be optimized in any way, both sets will have to be balanced by the process. We now know that we were in search of a logic map that can handle both sets of constraints simultaneously. Information is all about possibilities, and information systems, such as the genetic code, are all about relationships between possibilities that we call logic. Those skilled in the art will appreciate that nature took the track of starting with four possibilities and expanded forward to near infinity in a non-linear fashion at least in part by leveraging the logic and symmetry of the dodecahedron. The conventional wisdom in the past is to understand how nature could squeeze sixty-four into twenty, when nature actually was moving from one to four to twenty and beyond.

### INDUSTRIAL APPLICABILITY

Maps according to the present invention allow one organize information regarding related sets of things, such as in computer code or an electronic database, or to view relationships among two sets of symbols. A map according to the present invention can be as simple as one of the triangles of FIG. 5, or as complex as the complete generalized map of FIG. 2. Preferably, although not necessarily, the map is presented on a visible globe, or a computer display equivalent, which includes but is not limited to spheres, dodecahedrons, icosahedrons, “soccer balls”, and the like. When the generalized map is applied to the genetic code, previously unseen patterns emerge. Six of the triangles of FIG. 5 include three members from the first set of symbols (the colored stars representing nucleic acid base codes), and from one to six members from the second set of symbols (codons or amino acids and stops). When the first set of symbols are constrained to being one of four different types, the triangles assume one of a primary pattern, a secondary pattern and a tertiary pattern, as shown by rows 1, 2-4 and 5 of FIGS. 5 and 7, respectively.

In one aspect, symbols according to the present invention can be thought of as being distributed according to points, edges and faces of regular solids. Although the present invention has been illustrated using symbols such as color, points as the intersection of faces, regions outlined by lines, odd shaped regions representing a single amino acid, words, letters, numbers, or even variables in computer code or an electronic database, symbols according to the present invention can take on any suitable form. In other words, the invention is not so much concerned with what symbols are chosen, only that they be networked with one another as per the illustrated maps. Although much of the invention has been illustrated in the context of a map having three each of four genetic base codes, those skilled in the art will appreciate that other pattern maps could be created with an unequal distribution of genetic base codes. It is this aspect of the invention that can be used to explain codon bias as a function of GC content. Although the present invention has been illustrated with color coding the amino acids according to water infinity, those skilled in the art will appreciate that other properties, or a mixture thereof, could be represented through color symbols. For instance, other symbols, which may include color could be used to see what patterns emerge by assigning symbols to the molecular weight, size of the amino acid molecules, flexibility of bonds, types of bonds or any other property that can be expressed in relative terms among or between amino acids. Although the present invention finds particular applicability in mapping relationships among nucleic acids and amino acids, those skilled in the art will appreciate that the Rafiki model could find other applications as well, such as in physics, and in quantum mechanics in particular. Thus, the present invention could find potential application in any system exhibiting dodecahedral logic.

The following is a list of observed phenomena that the present invention assists in explaining:

1. Synonymous codons are not always functionally synonymous.

2. Codons require context.

3. Some codon combinations cannot be translated within a genome.

4. GC content drives codon usage.

5. Codons can disappear entirely from genomes.

6. TRNA populations vary between genomes.

7. Codon usage and TRNA expression is correlated.

8. Codons can specify more that one TRNA within a genome.

9. One TRNA can recognize more than one codon.

10. TRNA molecules are not homogenous with or between genomes.

11. Xenogentic sequences produce translation difficulties.

12. Synonymous mutations can alleviate xenogenic translation difficulties.

13. Primary structure determines tertiary structure in proteins.

14. Primary sequence analysis has failed to accurately predict secondary structure.

The genetic code is part of a complex crystallization process we call life. The currently accepted linear model holds that the genetic code is a one dimensional, sequential, non-overlapping relationship between nucleic acids and amino acids. This model has proven insufficient in explaining the multi-dimensional process of translation between nucleic acids and amino acids. An alternative to the linear model proposed here, the Rafiki Model of the genetic code, differs from the currently accepted one in three important ways.

1. The genetic code embodies two fundamental forms of information regarding translation. First, it carries information about the stereo-chemistry of peptide bonds. Second, it carries amino acid sequence information.

The primary structure of the poly-peptide results from the information contained in the genetic code. The amino acid sequence of a polypeptide is merely a subset of the total information translated from the nucleic acid sequence.

2. The genetic code has a geometric foundation of coincident symmetry from all five regular solids. Information in the system is based primarily on the symmetry relationships between two regular solids; the tetrahedron and the dodecahedron.

Erwin Schrodinger proposed that life is an aperiodic crystal. He was essentially correct, but every repeatable crystal structure requires a simple repeatable symmetry to enable consistent construction of molecular morphology. Aperiodic crystals are by definition not constructed on repeating symmetry, and therefore a simple map directing consistent morphology is difficult to imagine. However, a map of the symmetry relationship between shapes can generate tremendous complexity, and for all practical purposes this relationship functions in a simple, aperiodic way.

The genetic code is based on the interaction of symmetries, essentially mapping the relationships between them. The genetic language is a language of shapes, primarily translating dodecahedrons into tetrahedrons.

3. The genetic code is a hierarchical system of combinatorial, molecular elements. Nucleic acids are the base element in the system. These combine in triplets (codons) to specify a TRNA molecule. The TRNA molecules combine, possibly in quartets (peptones), to define a peptide bond. Peptide bonds combine to define the primary structure of proteins.

The primary sequence of proteins can be determined by examining either the primary structure of the polypeptides or the sequence of nucleic acids, but the peptide bonds cannot be determined by examining a nucleic acid sequence alone. Only by examining the combination of TRNA molecules in a pepton can the peptide bond be determined from the code.

Therefore, the complete genetic code in an organism is a system that must conceptually include MRNA and TRNA. Ribosomal RNA participates by providing a structural base for MRNA during translation, as well as providing the enzymatic activity of peptide bond formation. In this way, RRNA might be viewed as an active voice in the genetic code as well. A protein's primary structure is the fundamental output of the genetic code, and amino acids are the mono-numeric units of that output.

The Rafiki model assimilates these new conceptual elements into a model of the genetic code. This model provides a more robust and accurate understanding of the complex crystallization process we know as life. From this perspective, we can recognize that variation in the nature of TRNA populations from one organism to another naturally occurs, and therefore the system is no longer constrained to universality. The relationships between regular solids, however, are universal.

The Rafiki Model helps explain many of the perplexing phenomena being discovered today at an ever-accelerating pace, phenomena that cannot be explained adequately by the linear model.

Another potential use of the invention could be as a decoder and/or encoder. The following is an example of a code based on the general Rafiki model of FIG. 2. If we begin by assigning the capitol letters A-L to the numerical values 1-12, then we can assign the following symbols to the variables for A and B.

| Variable | Symbol | | | | | |
|---|---|---|---|---|---|---|
| A1 | A | | | | | |
| A2 | B | | | | | |
| A3 | C | | | | | |
| A4 | D | | | | | |
| A5 | E | | | | | |
| A6 | F | | | | | |
| A7 | G | | | | | |
| A8 | H | | | | | |
| A9 | I | | | | | |
| A10 | J | | | | | |
| A11 | K | | | | | |
| A12 | L | | | | | |
| | Permutation | Permutation | Permutation | Permutation | Permutation | Permutation |
| Variable | 1 | 2 | 3 | 4 | 5 | 6 |
| B1 | AA | AB | AC | AD | AE | AF |
| B2 | BA | BB | BC | BD | BE | BF |
| B3 | CA | CB | CC | CD | CE | CF |
| B4 | DA | DB | DC | DD | DE | DF |

The triplets for the C variables are dictated by the relationships within the dodecahedron, as shown in FIG. 2. Any appropriate means can be assigned to the C symbols or variables. Furthermore, continued hierarchies of symbol relationships are possible, and entirely new sets can be created, overlapped and layered, as does nature in the genetic code.

| Variable | Triplet | Symbol | Variable | Triplet | Symbol | Variable | Triplet | Symbol |
|---|---|---|---|---|---|---|---|---|
| C1 | AFB | d | C41 | IBF | c | C81 | EJK | v |
| C2 | ABC | z | C42 | IFJ | i | C82 | EFJ | o |
| C3 | ACD | k | C43 | IJL | j | C83 | EAF | 6 |
| C4 | ADE | y | C44 | ILH | SPACE | C84 | EDA | i |
| C5 | AEF | u | C45 | IHB | o | C85 | EKD | x |
| C6 | BIH | k | C46 | JFE | c | C86 | FIJ | STOP |
| C7 | BHC | Capitol | C47 | JEK | y | C87 | FBI | p |
| C8 | BCA | a | C48 | JKL | 1 | C88 | FAB | Capitol |
| C9 | BAF | q | C49 | JLI | b | C89 | FEA | h |
| C10 | BFI | o | C50 | JIF | START | C90 | FJE | y |
| C11 | CHG | c | C51 | KED | m | C91 | GCD | a |
| C12 | CGD | STOP | C52 | KDG | t | C92 | GHC | n |
| C13 | CDA | t | C53 | KGL | a | C93 | GLH | a |
| C14 | CAB | i | C54 | KLJ | Capitol | C94 | GKL | m |
| C15 | CBH | b | C55 | KJE | k | C95 | GDK | i |
| C16 | DGK | u | C56 | LKG | e | C96 | HBC | i |
| C17 | DKE | a | C57 | LGH | l | C97 | HIB | f |
| C18 | DEA | SPACE | C58 | LHI | e | C98 | HLI | m |
| C19 | DAC | e | C59 | LIJ | j | C99 | HGL | d |
| C20 | DCG | v | C60 | LJK | r | C100 | HCG | STOP |
| C21 | EKJ | n | C61 | ABF | . | C101 | IGB | w |
| C22 | EJF | d | C62 | ACB | o | C102 | IJF | m |
| C23 | EFA | j | C63 | ADC | i | C103 | ILJ | 1 |
| C24 | EAD | p | C64 | AED | t | C104 | IHL | z |
| C25 | EDK | f | C65 | AFE | q | C105 | IBH | SPACE |
| C26 | FJI | e | C66 | BHI | b | C106 | JEF | 1 |
| C27 | FIB | RE- | C67 | BCH | ; | C107 | JKE | p |
| | | TURN | | | | | | |
| C28 | FBA | g | C68 | BAC | y | C108 | JLK | 2 |
| C29 | FAE | a | C69 | BFA | w | C109 | JIL | v |
| C30 | FEJ | . | C70 | BIF | w | C110 | JFI | h |
| C31 | GDC | s | C71 | CGH | r | C111 | KDE | SPACE |
| C32 | GCH | 3 | C72 | CDG | . | C112 | KGD | o |
| C33 | GHL | g | C73 | CAD | 5 | C113 | KLG | 4 |
| C34 | GLK | f | C74 | CBA | — | C114 | KJL | x |
| C35 | GKD | t | C75 | CHB | u | C115 | KEJ | e |
| C36 | HCB | u | C76 | DKG | e | C116 | LGK | 8 |
| C37 | HBI | s | C77 | DEK | l | C117 | LHG | g |
| C38 | HIL | 9 | C78 | DAE | h | C118 | LIH | RE- |
| | | | | | | | | TURN |
| C39 | HLG | i | C79 | DCA | RE- | C119 | LJI | 0 |
| | | | | | TURN | | | |
| C40 | HGC | Capitol | C80 | DGC | 7 | C120 | LKJ | h |

The coded message can be preceded by any sequence of “nonsense” symbols. A legitimate reading frame is established when the START symbols are encountered. In the C layer the START symbols are the string of three symbols “JIF”. In the B layer the START symbols are the string of two symbols “OB”. The following message can be encoded with C variables as follows.

Imagination is more important than knowledge—Albert Einstein.

TJEDGEDSODLKDJIFFABIFJHLIDKEGHLHBCEKJFAEAEDEDAIHBG
HCILHHBCGDCDEAGKLBFICGHLHIKDEIFJKEDFBIEFJLJKCDAFAE
EKJKDGIBHGKDDAEGLHGHCDEAKJEGHCBFIBFALGHDACEJFGHLDK
GILHCBAKDEKLJBCALGHCBHLKGLJKCDAILHHGCLHIADCGHCGDCK
DGDACHLGGHCCDGLIH

Those skilled in the art will appreciate that the present invention could take on a wide variety of forms apart from those illustrated. For instance, a map of the present invention could be rendered in a virtual computer space such as being embedded in programming code or contained in an electronic database, without departing from the intended scope of the present invention. Although the invention has been illustrated as a decoding device for an arbitrary code, those skilled in the art will recognize that the same principals could be applied to decoding the genetic code into one of codons, amino acids and TRNA or even a stereochemical polypeptide chain. The genetic code is cast as a sequence of twelve symbols that are each linked to a plurality of different codons, amino acids and TRNA. Those skilled in the art will appreciate that codons actually designate TRNA, not amino acids. Therefore, each amino acid in the maps could also be a symbol representing the specific TRNA that designates it. The present invention could also be used as a way to demonstrate the specific genetic code of a given organism. This could be accomplished by starting with the map of the present invention, determining the population of TRNA for that organism, and then mapping that population onto the globe of the present invention. In fact, such a mapping could be used to demonstrate the relationships among organisms on the planet earth. This mapping could also be used to demonstrate why a string of DNA can be processed by one organism but not another, because its TRNA population is incompatible with certain DNA sequences occurring in another organism. By using peptide bond configuration data, the present invention will facilitate the translation of genetic base codes into amino acid assignments and the stereochemical configuration of the peptide bond between adjacent amino acids. This would enable one to decode sequences of DNA into the primary structure of a protein, which dictates function through the secondary and tertiary structures. Sequences to be translated can have 50 or fewer members, or a sequence of 500 or more, possibly reflecting an entire protein. Although the preffered version of the invention includes the complete network of relationships using twelve nucleic acids, more nucleic acids could be used. For instance, the combined map of FIG. 5 has 60, but the unfolded icosahedron of FIG. 6 has 22. With a slight change, the icosahedron of FIG. 6 could utilize 23 nucleic acids, which is one less than the most compact grids of the prior art. Thus, the present invention could take on a variety of forms without departing from the intended scope of the invention which is defined in terms of the claims set forth below.

What is claimed is:

1. A method of decoding a code having a sequence of symbols, each symbol being from a first set of from 12-23 symbols, comprising the steps of:
linking the first set of symbols in a network to a second set of at least twenty translated symbols, wherein each member of the first set of symbols is linked to several members of the second set of translated symbols; and
translating the sequence of symbols into a sequence of translated symbols using the network.

2. The method of claim 1 wherein the first set of symbols represent nucleic acids; and
the second set of translated symbols represent one of codons, TRNA and amino acids.

3. The method of claim 1 in which there are twelve symbols in the first set.

4. The method of claim 3 wherein the twelve symbols represent nucleic acids; and
the translated symbols represent one of codons, TRNA and amino acids.

5. The method of claim 4 including a step of organizing the network into the equivalent of a icosahedron and a dodecahedron.

6. The method of claim 1 in which the sequence is at least 50 symbols long.

7. The method of claim 6 in which the sequence is at least 500 symbols long.

8. A map comprising:
a first set of symbols and a second set of symbols;
a set of less than seven members of said second set of symbols being mapped to a set of three members of said first set of symbols; and
relationships between said first and second sets of symbols representing relationships between genetic base codes and amino acids that occur in nature.

9. The map of claim 8 wherein said first set of symbols have at least twelve but less than twenty four members representing genetic base codes; and
said second set of symbols representing twenty amino acids and at least one stop.

10. The map of claim 9 wherein said symbols are on a substrate that includes a globe; and
said first set of symbols has twelve members representing four genetic base codes.

11. The map of claim 10 wherein each member of said set of three members being identifiably different from a remaining two.

12. The map of claim 9 wherein said second set of symbols include a plurality of colors; and
said plurality of colors including a representation of a relative property among said amino acids.

13. The map of claim 12 wherein said relative property includes water affinity.

14. The map of claim 9 wherein each of said amino acids being represented by different contiguous regions on a substrate.

15. The map of claim 9 wherein said symbols appear on a two dimensional substrate; and
said first and second sets of symbols having a pattern corresponding to one of an unfolded dodecahedron and an unfolded icosahedron.

16. A map comprising:
twenty assignments mapped to subsets of twenty amino acids and stops;
four of said subsets having a primary pattern;
twelve of said subsets having a secondary pattern; and
four of said subsets having a tertiary pattern.

17. The map of claim 16 wherein each said primary pattern represents one of four amino acids;
each said secondary pattern representing at least two, but no more than three, different amino acids; and
each said tertiary pattern representing at least four, but no more than six different amino acids.

18. The map of claim 17 including symbols that define contiguous areas on a substrate;
each of said contiguous areas having a color and being representative of one amino acid; and
each said color representing a relative property among said amino acids.

19. The map of claim 18 wherein said relative property includes water affinity.

20. The map of claim 17 wherein each of said twenty assignments has three vertices on a substrate; and
each of said vertices represent a genetic base code that is shared by five of said twenty assignments.

21. The map of claim 20 wherein said map includes twelve identifiably different vertices; and
said twelve identifiably different vertices representing genetic base codes.

22. The map of claim 16 on a substrate that includes a globe;
said four primary pattern subsets are distributed on said globe in a first tetrahedral relationship; and
said four tertiary pattern subsets are distributed on said globe in a second tetrahedral relationship.

23. The map of claim 22 wherein said first tetrahedral relationship and second tetrahedral relationship are duals of one another.

24. The map of claim 16 wherein each primary pattern subset is contiguous with a set of three secondary pattern subsets; and
each tertiary pattern subset is contiguous with another set of three secondary pattern subsets; and
each of said secondary pattern subsets is contiguous with one primary pattern subset and one tertiary pattern subset.

25. A map comprising:
at least twenty subsets mapped to each other in a network of relationships;
each of said subsets being representative of one of twenty amino acids and stops; and
at least one of said subsets representing an amino acid corresponding to a plurality of codons.

26. The map of claim 25 wherein said subsets are represented by symbols distributed on a globe.

27. The map of claim 26 wherein said symbols include colors representing a relative property among said twenty amino acids.

28. The map of claim 27 wherein said relative property includes water affinity.

29. The map of claim 25 wherein at least one of said subsets representing an amino acid corresponding to a plurality of base code codons.

30. The map of claim 25 including a set of symbols uniformly distributed on a substrate, and representing twelve genetic base codes.

31. The map of claim 25 wherein a plurality of said subsets represent a same amino acid.

32. A map comprising:
a first set of symbols mapped to a second set of symbols;
said first set of symbols including less than twenty four members, which are each one of four different types;
said second set of symbols including at least twenty different members; and
each combination of three members of said first set of symbols being mapped to at least one member of said second set of symbols;
at least one said combination being mapped to a plurality of different members of said second set of symbols.

33. The map of claim 32 including three members of said first set of symbols arranged to define a triangle containing at least one, but less than seven, different members of said second set of symbols;
said first set of symbols representing genetic base codes;
said second set of symbols representing amino acids; and
said first and second sets of symbols being related according to codon-amino acid assignments that occur in nature.

34. The map of claim 33 including twenty of said triangles.

35. The map of claim 34 wherein five of said triangles share a common vertex.

36. The map of claim 35 wherein different ones of said twenty triangles have a primary pattern, a secondary pattern and a tertiary pattern.

37. A method of determining an assignment relationship between genetic base codes and amino acids, comprising the steps of:
mapping a network of relationships among genetic base codes and amino acids;
identifying one of an amino acid and an ordered group of three base codes in said network;
reading from said network one of, an ordered group of three base codes mapped to said amino acid, and an amino acid mapped to said ordered group of three base codes.

38. A map comprising:
genetic base codes arranged in a pattern corresponding to at least a portion of a regular solid; and
amino acids mapped in a predetermined relationship with respect to said genetic base codes such that each ordered combination of three genetic base codes are mapped to one of said amino acids; and
said predetermined relationship reflecting a genetic base code-amino acid assignment relationship that occurs in nature.
